(19) United States
(12) Patent Application Publication
(10) Pub. No.: US 2025/0228610 A1
(43) Pub. Date: Jul. 17, 2025

(54) ENDOSCOPE TREATMENT TOOL

(71) Applicant: Olympus Corporation, Tokyo (JP)

(72) Inventors: Yasuhito SUGA, Musashino-shi (JP); Yuta MUYARI, Hino-shi (JP); Chika MIYAJIMA, Hachioji-shi (JP); Hiromasa KATO, Tokyo (JP); Takanobu YANAI, Hachioji-shi (JP)

(21) Appl. No.: 19/015,612

(22) Filed: Jan. 9, 2025

Related U.S. Application Data
(60) Provisional application No. 63/620,006, filed on Jan. 11, 2024.

(51) Int. Cl. A61B 18/14; A61B 1/00; A61B 18/00; A61B 90/00

(52) U.S. Cl. CPC A61B 18/1492; A61B 1/00087; A61B 90/03; A61B 2018/00077; A61B 2018/00083; A61B 2018/00184; A61B 2018/00982; A61B 2090/034

(57) ABSTRACT

An endoscope treatment tool can include a sheath, a conductive rod that protrudes from a distal end of the sheath, an electrode connected to a distal end of the rod, an insulator located distally relative to the electrode; and an insulating member that covers at least part of an outer circumferential surface of the rod.

### CROSS-REFERENCE TO RELATED APPLICATIONS

The present application claims priority based on U.S. Patent Provisional Application No. 63/620,006 filed in the United States on Jan. 11, 2024, the contents of which are incorporated herein by reference.

### TECHNICAL FIELD

This disclosure relates to an endoscope treatment tool and treatment method.

### BACKGROUND

In endoscope treatments such as ESD (endoscopic submucosal dissection), as shown in PCT International Publication No. WO 2014/061701 (Patent Document 1) and the like, endoscope treatment tools such as high-frequency knives can be used. A surgeon can use endoscope treatment tools such as high-frequency knives to perform incisions of biological tissues.

### SUMMARY

A high-frequency knife described in Patent Document 1 and the like is suitable for wide-area incisions, but can be unsuitable for restricting incisions to a narrow area.

The present disclosure provides an endoscope treatment tool that can suitably perform incisions on biological tissue.

An endoscope treatment tool according to the first aspect of the present disclosure can include a sheath, a conductive rod protruding from the distal end of the sheath, an electrode connected to the distal end of the rod, an insulator provided distal to the electrode, and an insulating member covering at least part of the outer circumferential surface of the rod.

The endoscope treatment tool of the present disclosure can suitably perform incisions on biological tissue.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, various embodiments discussed in the present document, but the embodiments are not limited to these examples.

FIG. 1 is an example of an overall view of an endoscope treatment system according to a first embodiment.

FIG. 2 is an example of an overall view showing a treatment tool.

FIG. 3 is an example of a perspective view of a distal end portion of the treatment tool.

FIG. 4 is an example of a side view of the distal end portion of the treatment tool.

FIG. 5 is an example of a side view of the distal end portion of the treatment tool.

FIG. 6 is an example of a cross-sectional view taken along line X1-X1 in FIG. 4.

FIG. 7 is an example of a cross-sectional view taken along line X2-X2 in FIG. 6.

FIG. 8 is an example of a perspective view of the distal end portion of the treatment tool as viewed from the proximal end side.

FIG. 9 is an example of a diagram showing a modified insulating member.

FIG. 10 is an example of a cross-sectional view of the modified insulating member.

FIG. 11 is an example of a cross-sectional view taken along line X3-X3 in FIG. 10.

FIG. 12 is an example of a diagram showing another modified insulating member.

FIG. 13 is an example of a cross-sectional view of the modified insulating member.

FIG. 14 is an example of a cross-sectional view taken along line X4-X4 in FIG. 13.

FIG. 15 is an example of a cross-sectional view taken along line X5-X5 in FIG. 13.

FIG. 16 is an example of a diagram showing a modified insulating chip.

FIG. 17 is an example of a side view of the distal end of a treatment tool according to a second embodiment.

FIG. 18 is an example of a cross-sectional view of the distal end of the treatment tool.

FIG. 19 is an example of a cross-sectional view showing a modified insulating member.

FIG. 20 is an example of an overall view of a treatment tool according to a third embodiment.

FIG. 21 is an example of a cross-sectional view of the distal end of the treatment tool.

FIG. 22 is an example of a cross-sectional view of the distal end of the treatment tool.

FIG. 23 is an example of a cross-sectional view of the distal end of the treatment tool.

FIG. 24 is an example of a cross-sectional view taken along line Y1-Y1 in FIG. 21.

FIG. 25 is an example of a cross-sectional view taken along line Y2-Y2 in FIG. 21.

FIG. 26 is an example of a cross-sectional view taken along line Y3-Y3 in FIG. 21.

FIG. 27 is an example of a cross-sectional view taken along line Y4-Y4 in FIG. 21.

FIG. 28 is an example of a view showing the movement of the knife caused by the operation of the operating portion.

FIG. 29 is an example of a view showing the movement of the knife caused by the operation of the operating portion.

FIG. 30 is an example of a diagram showing the movement of the knife when the operating portion is operated.

FIG. 31 is an example of a diagram showing the movement of the knife when the operating portion is operated.

### DETAILED DESCRIPTION

### First Embodiment

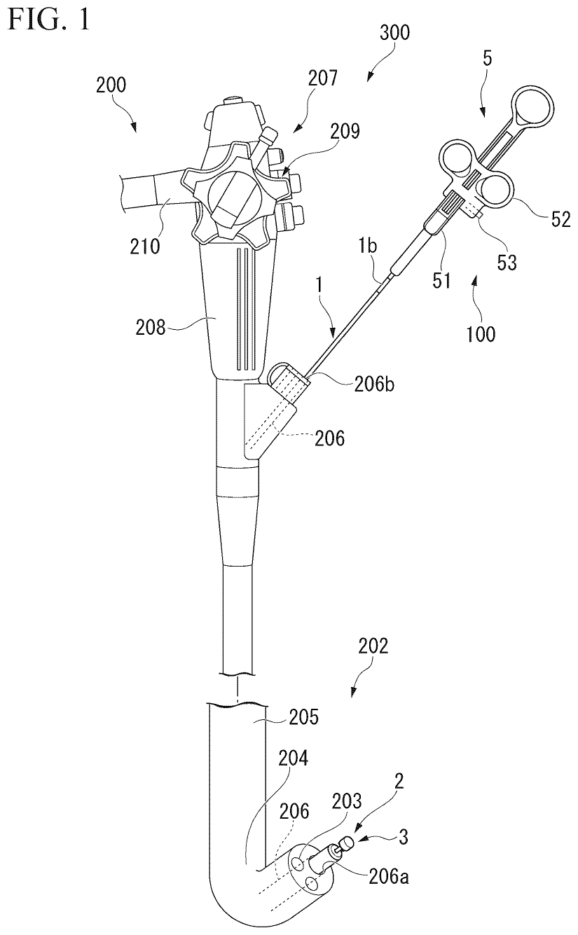

An endoscope treatment system 300 according to a first embodiment of the present disclosure will be described with reference to FIGS. 1 to 8. FIG. 1 is an example of an overall view of the endoscope treatment system 300 according to this embodiment.

### Endoscope Treatment System 300

As shown in FIG. 1, the endoscope treatment system 300 can include an endoscope 200 and a treatment tool 100. The treatment tool 100 can be inserted into the endoscope 200 for use.

### Endoscope 200

The endoscope 200 can be a flexible endoscope and can include an insertion portion 202 that can be inserted into the body from the distal end, and an operating portion 207 attached to the proximal end of the insertion portion 202.

The insertion portion 202 can include an imaging portion 203, a bending portion 204, and a flexible portion 205. The imaging portion 203, the bending portion 204, and the flexible portion 205 can be arranged in this order from the distal end of the insertion portion 202. Inside the insertion portion 202, a channel 206 can be provided for inserting the treatment tool 100. A distal end opening 206a of the channel 206 can be provided at the distal end of the insertion portion 202.

The imaging portion 203 can be equipped with or include an imaging element such as a charge-coupled device (CCD) or a complementary metal oxide semiconductor (CMOS), or any similar device capable of capturing an image of the area to be treated. The imaging portion 203 can capture an image of the distal end of the treatment tool 100 when the treatment tool 100 protrudes from the distal end opening 206a of the channel 206.

The bending portion 204 can bend in response to the operation of the operating portion 207 by user or operator such as a surgeon. The flexible portion 205 can be or include a flexible tubular portion.

The operating portion 207 can be connected to the flexible portion 205. The operating portion 207 can include a grip 208, an input portion 209, a proximal end opening 206b of the channel 206, and a universal cord 210. The grip 208 is a portion that can be held by the surgeon, such as a handle. The input portion 209 can accept operation input for bending the bending portion 204.

The universal cord 210 can include a video signal line that can output the image captured by the imaging portion 203a to the outside. The video signal line can be connected to a display device such as a liquid crystal display via an image processing device equipped with a processor or the like.

### Treatment Tool 100

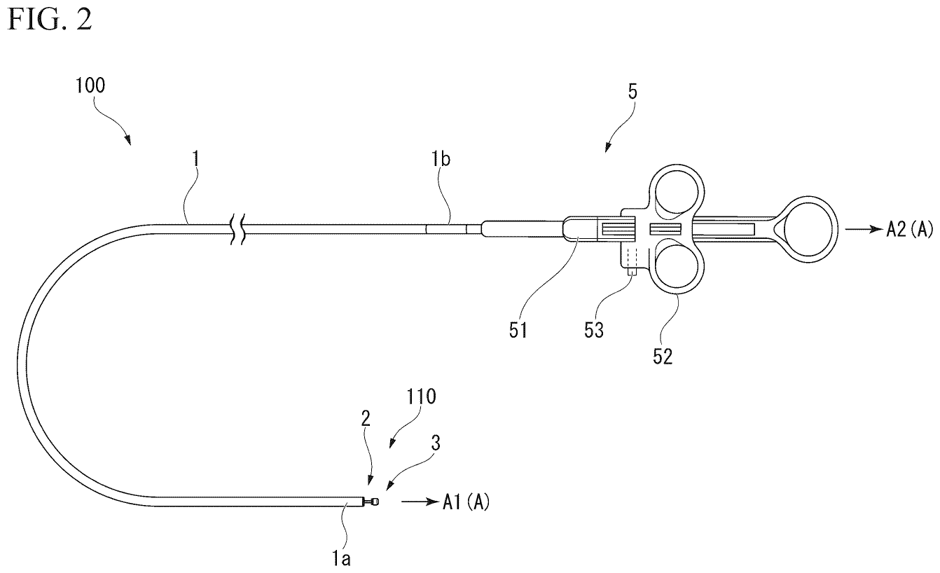

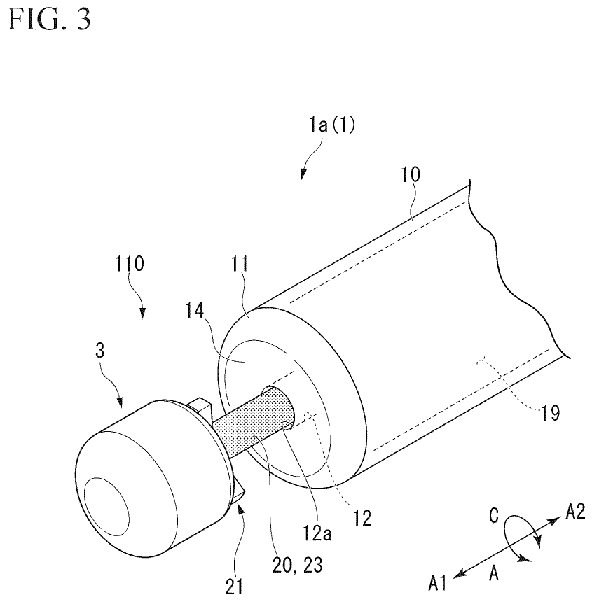

FIG. 2 is an example of an overall view showing the treatment tool 100. The treatment tool (endoscope treatment tool, high-frequency treatment tool) 100 can be an ESD knife. The treatment tool 100 can include a sheath 1, a knife 2, an insulating chip 3, an operation wire 4 (see FIG. 3), and a control portion 5. The knife 2 and the insulating chip 3 constitute a “treatment portion 110” that treats the affected area. In the following description, in the longitudinal axis direction (longitudinal direction, axial direction) A of the treatment tool 100, the side that is inserted into the patient's body is referred to as the “distal end side (distal side) A1” and the operating portion 5 side is referred to as the “proximal end side (proximal end side) A2”.

The sheath 1 can be or include a long tubular member extending from the distal end la to the proximal end 1b. The sheath 1 can be inserted into the channel 206 of the endoscope 200 and can advance and retract through the channel 206. As shown in FIG. 1, when the sheath 1 is inserted into the channel 206, the distal end la of the sheath 1 can protrude and retract from the distal end opening 206a of the channel 206.

FIG. 3 is an example of a perspective view of the distal end of the treatment tool 100. The sheath 1 can include an outer tube 10 extending in the longitudinal direction A, and a distal end member 11 provided at the distal end of the outer tube 10. The sheath 1 can be formed by integrally molding the outer tube 10 and the distal end member 11.

The distal end member 11 can be formed in a cylindrical shape. Note that “cylindrical” includes shapes close to a cylindrical shape (e.g., substantially cylindrical) in addition to a strictly cylindrical shape. The distal end member 11 can be formed of an insulating material such as resin. The distal end member 11 can include a through hole 12.

The through hole 12 can be a hole provided in the distal end member 11 and can pass through the distal end member 11 in the longitudinal axis direction A. The distal end of the through hole 12 can communicate with a first opening 12a formed in the distal end surface 14 of the distal end member 11. The proximal end of the through hole 12 can communicate with the internal space 19 of the outer tube 10.

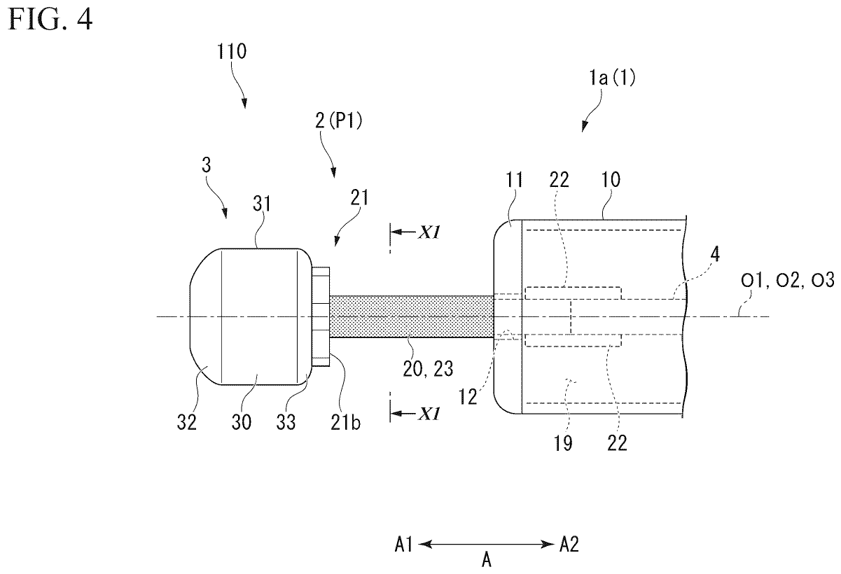

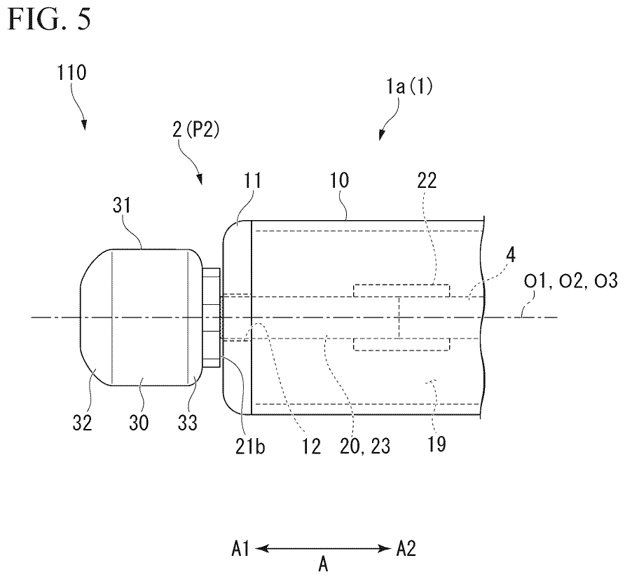

FIGS. 4 and 5 are example side views of the distal end of the treatment tool 100. The knife (electrode) 2 can be a metal member. The knife 2 can be made of a material such as stainless steel. The knife 2 can be conductive and can be energized with a high-frequency current. The knife 2 can include a rod 20, an electrode 21, a connector 22, and an insulating member 23.

The rod (blade, electrode body) 20 can be a metal round bar-shaped member. Note that “round bar-shaped” includes shapes close to a round bar shape (e.g., a substantially round bar shape) as well as a strict round bar shape. The rod 20 can be disposed on the distal end side A1 of the sheath 1. The operating wire 4 can be attached to the proximal end of the rod 20.

The rod 20 can be inserted through the through hole 12 of the distal end member 11 of the sheath 1 along the longitudinal axis direction A and can freely protrude and retract from the first opening 12a to the distal end side A1. The rod 20 can be fixed in a state where it protrudes from the first opening 12a to the distal end side A1 and cannot advance or retract.

The central axis O2 of the rod 20 in the longitudinal axis direction A can coincide with the central axis O1 of the sheath 1 in the longitudinal axis direction A. Note that “coinciding” includes a state in which they coincide strictly as well as a state in which they almost coincide (e.g., substantially coincide).

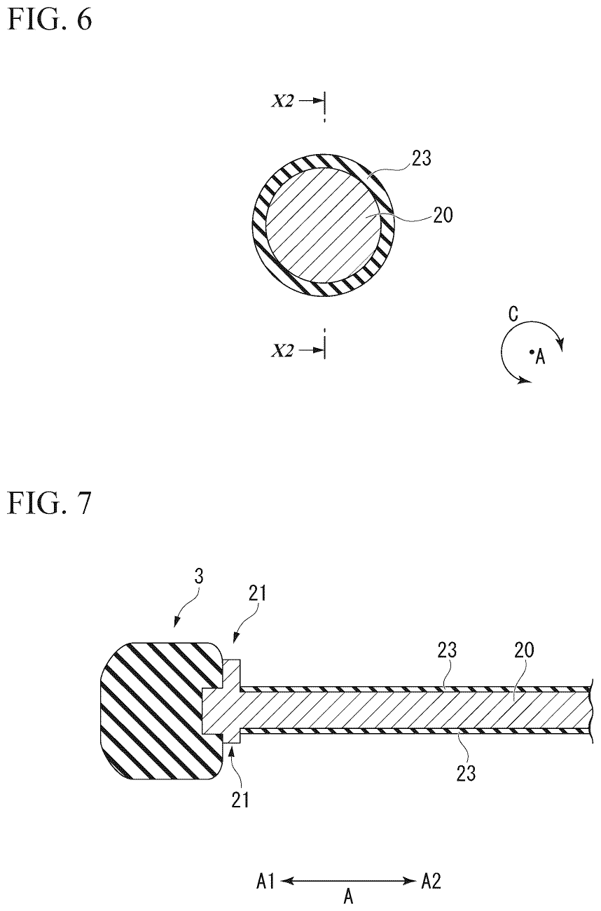

FIG. 6 is an example of a cross-sectional view taken along line X1-X1 in FIG. 4. FIG. 7 is an example of a cross-sectional view taken along line X2-X2 in FIG. 6. The outer peripheral surface of the rod 20 can be covered with an insulating member (second insulating member) 23.

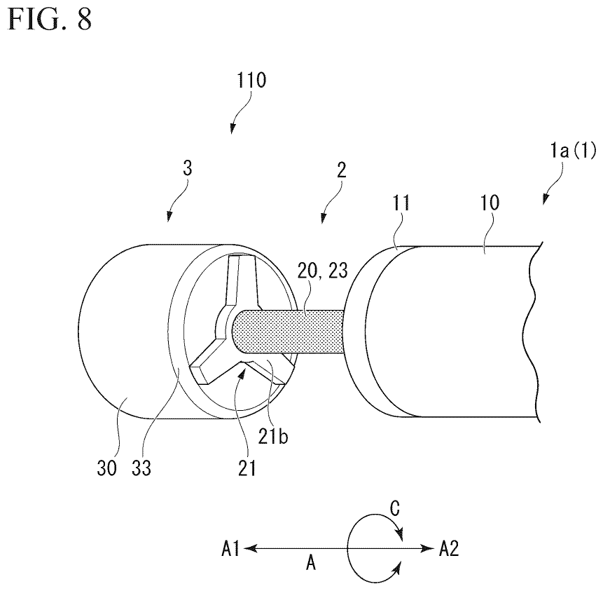

FIG. 8 is an example of a perspective view of the distal end of the treatment tool 100 as viewed from the proximal end side A2. The electrode (expanded diameter portion) 21 can be a plate-shaped conductive member connected to the distal end of the rod 20 and extending from the outer peripheral surface of the rod 20. The electrode 21 can extend in the radial direction of the longitudinal axis of the rod 20. A flat proximal end surface (back surface) 21b can be formed on the proximal end side A2 of the electrode 21. The proximal end surface 21 is not limited to a flat surface, and may be, for example, a surface with projections and recesses.

The electrodes 21 can protrude outward in the radial direction R from the outer peripheral surface of the rod. The electrodes 21 can extend at equal intervals along the circumferential direction C around the longitudinal axis of the rod 20. When viewed from the front in the direction along the longitudinal axis direction A, the electrodes 21 are formed in a trifurcated shape. The electrodes 21 may also be formed in a quadruple or pentapruned shape. That is, the electrodes 21 can be formed in a radial shape extending radially outward in the radial direction R from the central axis O2 of the rod 20. The electrodes 21 can also be formed in a flange shape such as a disk shape or a polygonal shape.

The insulating member 23 can cover the entire outer peripheral surface of the rod 20. In addition, the proximal end surface 21b of the electrode 21 and the distal end of the insulating member 23 can be aligned. That is, the insulating member 23 can cover at least the outer circumferential surface of the rod 20 from the proximal end of the electrode 21 to the distal end of the sheath 1. The insulating member 23 can be, for example, an insulating coating made of PTFE, PEEK, or the like.

The connector (connecting member) 22 can be a cylindrical member made of metal. Note that “cylindrical” includes shapes close to a cylindrical shape (e.g., substantially cylindrical) as well as a strict cylindrical shape. The connector 22 can connect the rod 20 and the operating wire 4.

As shown in FIG. 4, when the knife 2 is advanced relative to the sheath 1, the distal end of the connector 22 can come into contact with the distal end member 11. The contact between the distal end of the connector 22 and the distal end member 11 can position the knife 2 at the first position P1, which is the position on the most distal side A1.

As shown in FIG. 5, when the knife 2 is retracted relative to the sheath 1, the proximal end surface 21b of the electrode 21 can come into contact with the distal end surface 14 of the distal end member 11. In this example, the contact between the proximal end surface 21b of the electrode 21 and the distal end member 11 positions the knife 2 at the second position P2, which is the position on the most proximal side A2.

When the control wire 4 advances and retracts, the knife 2 can advance and retract from the first position P1 to the second position P2. The knife 2 can be fixed so that it cannot advance or retract while protruding from the first opening 12a to the distal end side A1.

A high-frequency current can be supplied to the knife 2 from the control wire 4 connected to the control portion 5. When a high-frequency current is supplied to the knife 2 from the control wire 4, the electrode 21 can function as a monopolar electrode that outputs a high-frequency current to the biological tissue. In this embodiment, since the outer circumferential surface of the rod 20 is covered with the insulating member 23, high-frequency current is not supplied to the biological tissue from the outer circumferential surface of the rod 20.

The insulating chip (insulator, first insulating member) 3 can be formed of an insulating material such as ceramic or resin. The insulating chip 3 can be provided on the distal end side A1 of the electrode 21. In this embodiment, the proximal end of the insulating chip 3 is fixed to the rod 20 in contact with the electrode 21. In the direction perpendicular to the longitudinal axis direction A, the electrode 21 can be disposed radially inward in the radial direction R of the outer surface 31 of the insulating chip 3 over the entire circumference.

The central axis O3 in the longitudinal axis direction A of the insulating chip 3 can coincide with the central axis O1 in the longitudinal axis direction A of the sheath 1. Note that “coincidence” includes a state in which the central axis O3 and the sheath 1 almost or substantially coincide with each other in addition to a state in which the central axis O1 and the sheath 1 coincide with each other strictly.

The insulating chip 3 can include a main body 30, a distal end portion 32 disposed on the distal end side A1 of the main body 30, and a proximal end portion 33 disposed on the proximal end side A2 of the main body 30. The distal end portion 32, the main body 30, and the proximal end portion 33 can be arranged and connected in the longitudinal axis direction A. The distal end portion 32 and the proximal end portion 33 can be integrally formed or may be formed by connecting separate members.

The main body 30 can be formed in a cylindrical shape. The central axis of the distal end portion 32 in the longitudinal axis direction A can coincide with the central axis O3.

The distal end portion 32 can be provided at the distal end of the main body 30. The distal end portion 32 can be formed in a hemispherical (dome) shape, and the diameter can decrease toward the distal end side A1. The shape of the distal end portion 32 is not limited to a hemispherical shape and may be, for example, a cylindrical shape.

The proximal end 33 can be provided at the proximal end of the main body 30. The proximal end 33 can be formed in a hemispherical (dome) shape, and the diameter can taper toward the proximal end side A2. The shape of the proximal end 33 is not limited to a hemispherical shape and may be cylindrical.

The electrode 21 can be located between the insulating chip (first insulating member) 3 and the insulating member (second insulating member) 23 in the longitudinal axis direction A. At least part of the electrode 21 can protrude outward in the radial direction R of the rod 20 beyond the outer periphery of the insulating member 23. The outer diameter of the insulating member (second insulating member) 23 can be smaller than the outer diameter of at least part of the insulating chip (first insulating member) 3.

The operating wire 4 can be a metal wire that passes through the internal space (pipe, lumen) 19 of the outer tube 10. The operating wire 4 can be formed of a material such as stainless steel. The distal end of the operating wire 4 can be connected to the rod 20, and the proximal end of the operating wire 4 can be connected to the operating portion 5.

As shown in FIGS. 1 and 2, the operating portion 5 can include an operating portion main body 51, a slider 52, and a power supply connector 53.

The distal end of the operating portion main body 51 can be connected to the proximal end 1b of the sheath 1. The operating portion main body 51 can include an internal space through which the operating wire 4 can be inserted. The operating wire 4 can pass through the internal space 19 of the outer tube 10 and the internal space of the operating portion main body 51 and can extend to the slider 52.

The slider 52 can be attached to the operating portion main body 51 so as to be movable along the longitudinal axis direction A. The proximal end of the operating wire 4 can be attached to the slider 52. The surgeon can advance and retract the slider 52 relative to the operating portion body 51, thereby advancing and retracting the operating wire 4 and the knife 2.

The power supply connector 53 can be fixed to the slider 52. The power supply connector 53 can be connected to a high-frequency power supply device and can be connected to the proximal end of the operating wire 4 via a conductive wire. The power supply connector 53 can supply high-frequency current supplied from the high-frequency power supply device to the rod 20 via the operating wire 4. In an example, the power supply connector 53 can be fixed to the operating portion body 51, not to the slider 52.

According to the treatment tool 100 of this embodiment, the rod 20 can be covered with an insulating member 23, so that the conductive part through which electricity can flow to the tissue can be made small. Therefore, the biological tissue to be incised can be restricted and incised suitably. By making it possible to make an incision only with the electrode 21 arranged between the insulating chip 3 and the insulating member 23, thermal invasion of the tissue can be suppressed.

The first embodiment of the present disclosure has been described above in detail with reference to the drawings, but the specific configuration is not limited to this embodiment, and design changes and the like within the scope of the gist of the present disclosure are also included. In addition, the components shown in the above-mentioned embodiment and modifications can be appropriately combined to form a configuration.

### Modification 1

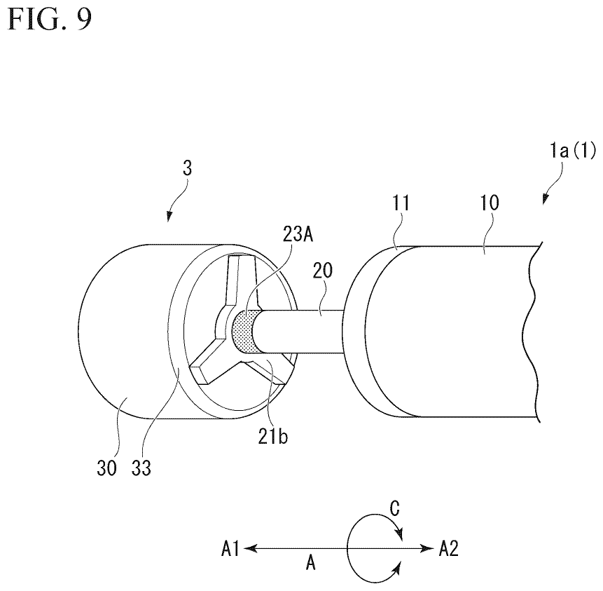

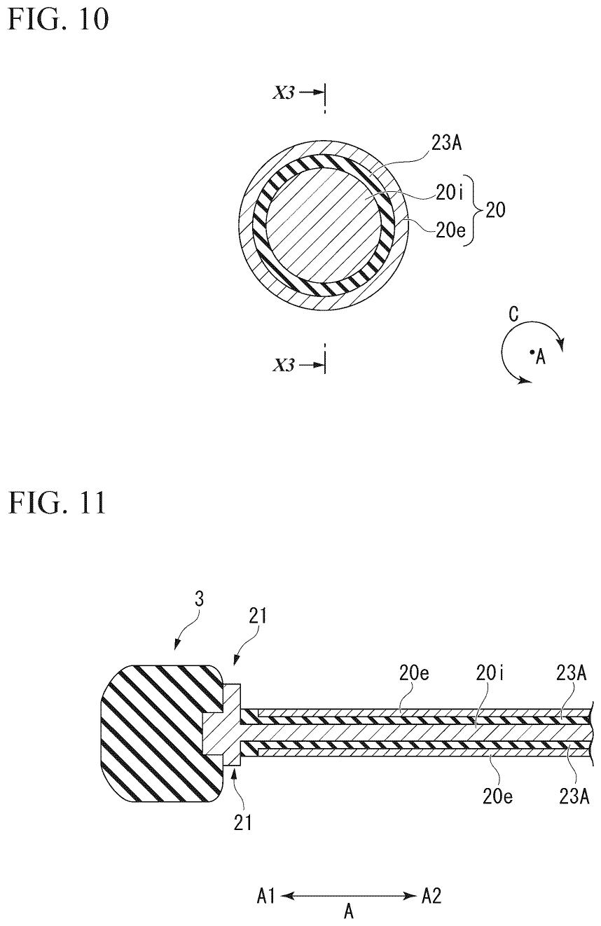

FIG. 9 is an example of a diagram showing an insulating member 23A which is a modification of the insulating member 23. FIG. 10 is an example of a cross-sectional view of the rod 20 and the insulating member 23A. FIG. 11 is an example of a cross-sectional view taken along the line X3-X3 shown in FIG. 10. The insulating member 23A can be provided inside the conductive rod 20. The rod 20 can include a rod core 20i and a rod outer tube 20e, and an insulating member 23A can be provided between the rod core 20i and the rod outer tube 20e. The rod core 20i can be connected to the electrode 21 and can supply high-frequency current to the electrode 21. The rod outer tube 20e can be made of a conductor but can be electrically isolated from the rod core 20i by the insulating member 23A, and, in such an example, the high-frequency current is not supplied to the rod outer tube 20e. Therefore, high-frequency current is not supplied to the biological tissue from the rod outer tube 20e.

### Modification 2

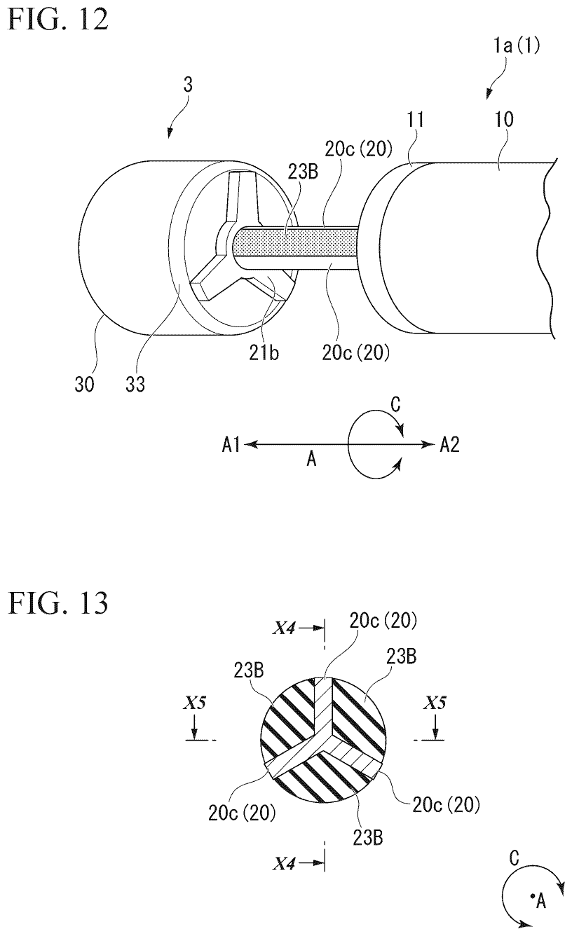

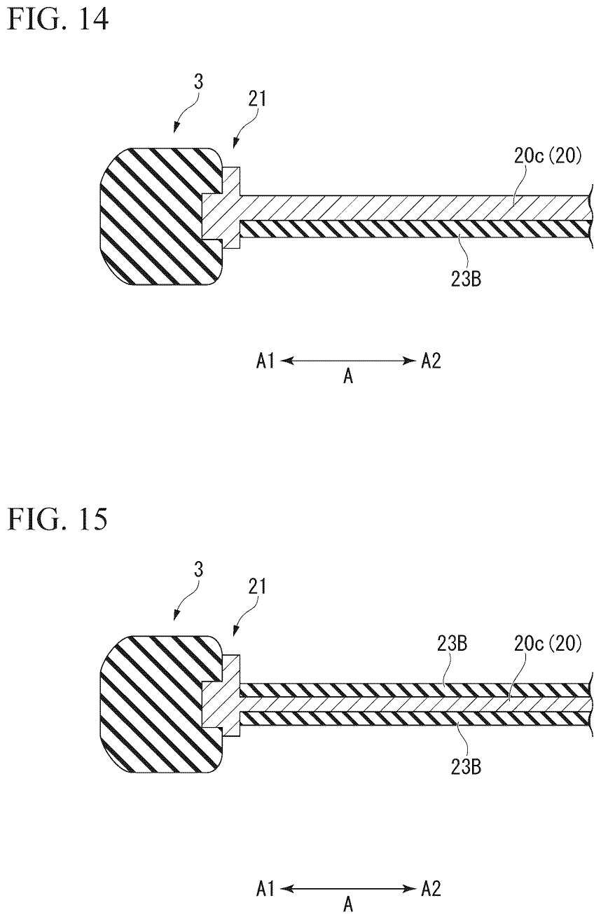

FIG. 12 is an example of a diagram showing an insulating member 23B, which is a modification of the insulating member 23. FIG. 13 is an example of a cross-sectional view of the rod 20 and the insulating member 23B. FIG. 14 is an example of a cross-sectional view taken along the line X4-X4 shown in FIG. 13. FIG. 15 is an example of a cross-sectional view taken along the line X5-X5 shown in FIG. 13. The insulating member 23B can cover part of the outer circumferential surface of the rod 20 in the circumferential direction C. Specifically, the insulating member 23B can intermittently cover a portion of the outer circumferential surface of the rod 20 in the circumferential direction C, and the insulating member 23B and the conductive region 20c can be alternately arranged in the circumferential direction C. The insulating member 23B can separate the outer circumferential surface of the rod 20 into multiple conductive regions such as three conductive regions 20c. The conductive regions 20c can extend along the longitudinal axis direction A and can have a rectangular shape. The insulating member 23B and the conductive regions 20c can be arranged along the outer circumferential surface of the rod 20, each covering half a circumference.

### Modification 3

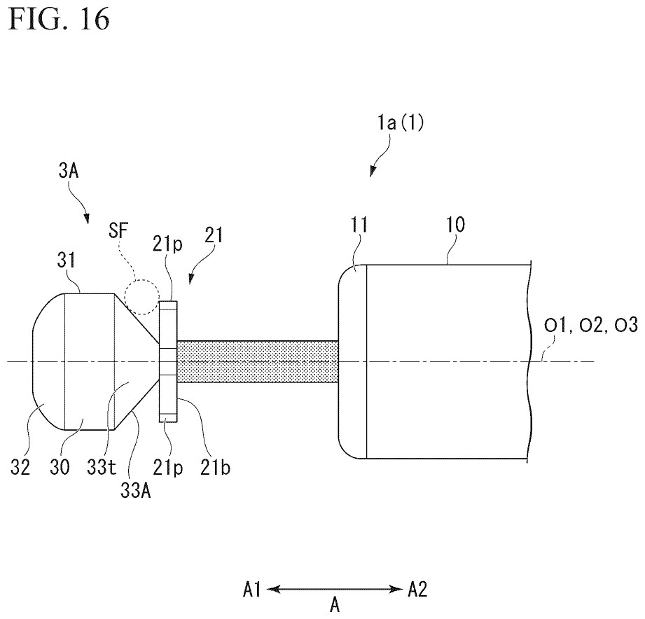

FIG. 16 shows an example of an insulating chip 3A, which is a modification of the insulating chip 3. The insulating chip 3A can include a main body 30, a distal end portion 32, and a proximal end portion 33A, which is a modification of the proximal end portion 33. The proximal end portion 33A can include a tapered surface 33t that narrows toward the electrode 21 (toward the proximal end side A2). The proximal end of the tapered surface 33t can abut against the base of the electrode 21. The electrode 21 can include a protruding portion 21p that can extend outward in the radial direction R of the rod 20 beyond the proximal end of the tapered surface 33t. The tapered surface 33t can be spaced from the protruding portion 21p, and the tapered surface 33t and the distal end surface of the protruding portion 21p can face each other with a gap therebetween, so that a front space (e.g., a gap) SF can be formed between the tapered surface 33t and the protruding portion 21p. By securing the front space (gap) SF of the protruding portion 21p of the electrode 21, the surgeon can perform an incision treatment using the electrode 21.

### Second Embodiment

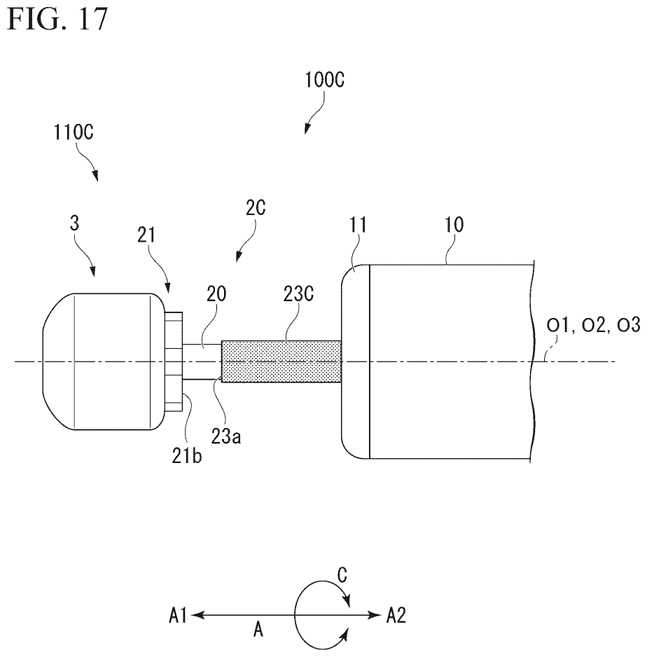

The treatment tool 100C according to the second embodiment of the present disclosure will be described with reference to FIGS. 17 to 19. In the following description, the same reference numerals are used for the configurations that are common to those already described, and duplicated descriptions will be omitted.

FIG. 17 is an example of a side view of the distal end of the treatment tool 100C. The treatment tool (endoscope treatment tool, high-frequency treatment tool) 100C can be an ESD knife. The treatment tool 100C can include a sheath 1, a knife 2C, an insulating chip 3, an operating wire 4, and an operating portion 5. The knife 2C and the insulating chip 3 can form a “treatment portion 110C” that treats the affected area.

The knife (electrode) 2C can be a metal member. The knife 2C can be made of a material such as stainless steel. The knife 2C can be conductive and a high-frequency current can be passed through it. The knife 2C can include a rod 20, an electrode 21, a connector 22, and an insulating member 23C.

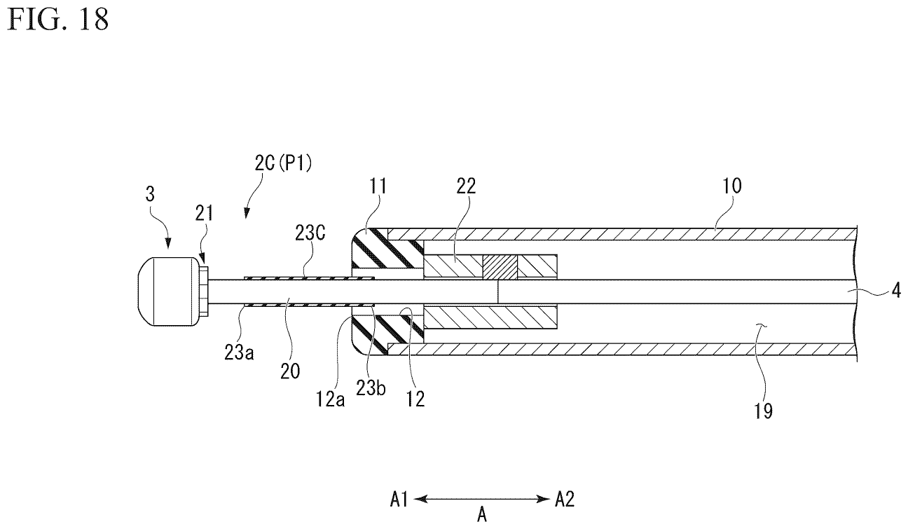

FIG. 18 is an example of a cross-sectional view of the distal end of the treatment tool 100C. The insulating member 23C can be an insulating tube that covers the outer periphery of the rod 20. The insulating member 23C can be an insulating tube made of, for example, PTFE or PEEK. The insulating member 23C can be attached to the rod 20 by fitting or thermal contraction. The distal end 23a of the insulating member 23C can be disposed on the proximal end side A2 of the electrode 21 in the rod 20, and a gap can be provided between the proximal end surface 21b of the electrode 21 and the distal end 23a of the insulating member 23C. The proximal end 23b of the insulating member 23C can be disposed on the distal end side A1 of the connector 22 that can connect the rod 20 and the operating wire 4. As a result, the distal end portion of the rod 20 can be exposed from the insulating member 23C. The insulating member 23C can cover the proximal end side of the rod 20 protruding from the sheath 1. The knife 2C can be configured so as not to pass electricity through at least part of the outer circumferential surface of the rod 20 to the biological tissue.

As shown in FIG. 18, the proximal end 23b of the insulating member 23C can be located on the proximal end side A2 of the first opening 12a of the sheath 1 and does not protrude from the first opening 12a, even when the knife 2 is located at the first position P1.

According to the treatment tool 100C of this embodiment, incision of biological tissue can be performed. By making it possible to perform incision with the exposed distal end portion of the electrode 21 and the rod 20, thermal invasion of the tissue can be suppressed.

Although the second embodiment of the present disclosure has been described above in detail with reference to the drawings, the specific configuration is not limited to this embodiment, and design changes and the like that do not deviate from the gist of the present disclosure are also included. In addition, the components shown in the above-mentioned embodiment and modifications can be appropriately combined and configured.

### Modification 2-1

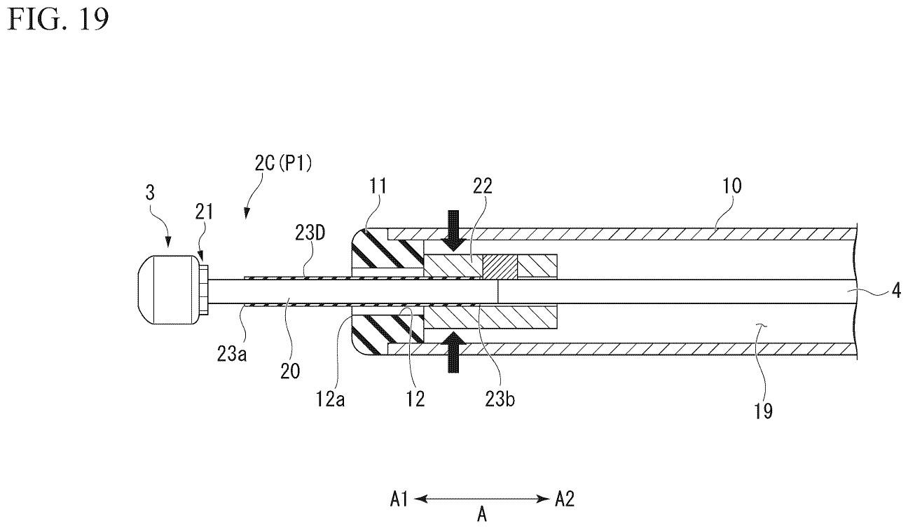

FIG. 19 is an example of a cross-sectional view showing an insulating member 23D, which is a modification of the insulating member 23C. The proximal end 23b of the insulating member 23D can extend to the connector 22. In this case, the proximal end 23b of the insulating member 23D can be crimped and fixed to the connector 22.

The insulating chip 3 of the treatment tool 100C can have a tapered surface 33t, as in the insulating chip 3A shown in FIG. 16. A front space (a gap) SF can be formed between the tapered surface 33t and the protruding portion 21p of the electrode 21.

### Third Embodiment

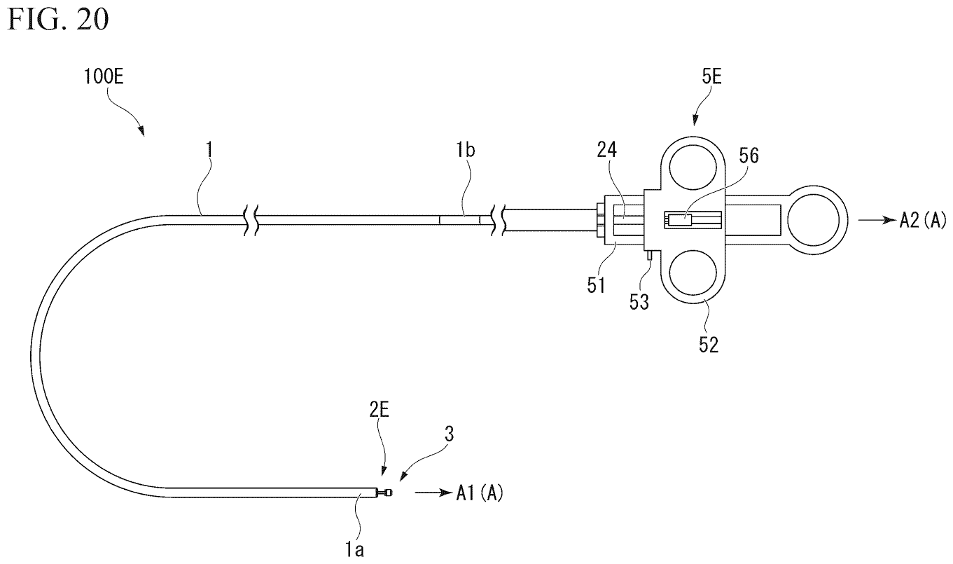

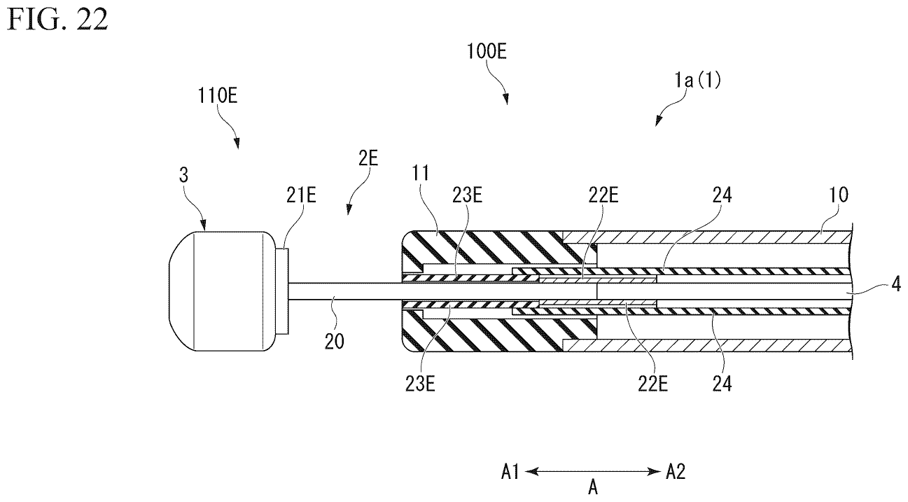

A treatment tool 100E according to a third embodiment of the present disclosure will be described with reference to FIGS. 20 to 27. In the following description, components common to those already described will be denoted by the same reference numerals and duplicated description will be omitted.

FIG. 20 is an example of an overall view showing the treatment tool 100E. The treatment tool (endoscope treatment tool, high-frequency treatment tool) 100E can be an ESD knife. The treatment tool 100C can include a sheath 1, a knife 2E, an insulating chip 3, an operating wire 4, and an operating portion 5E. The knife 2E and the insulating chip 3 can constitute a “treatment portion 110E” that treats the affected area.

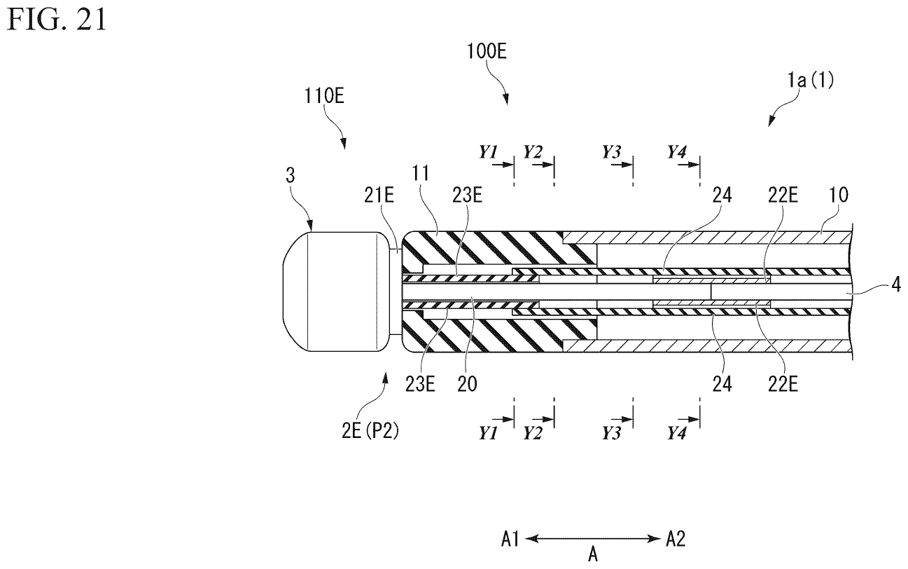

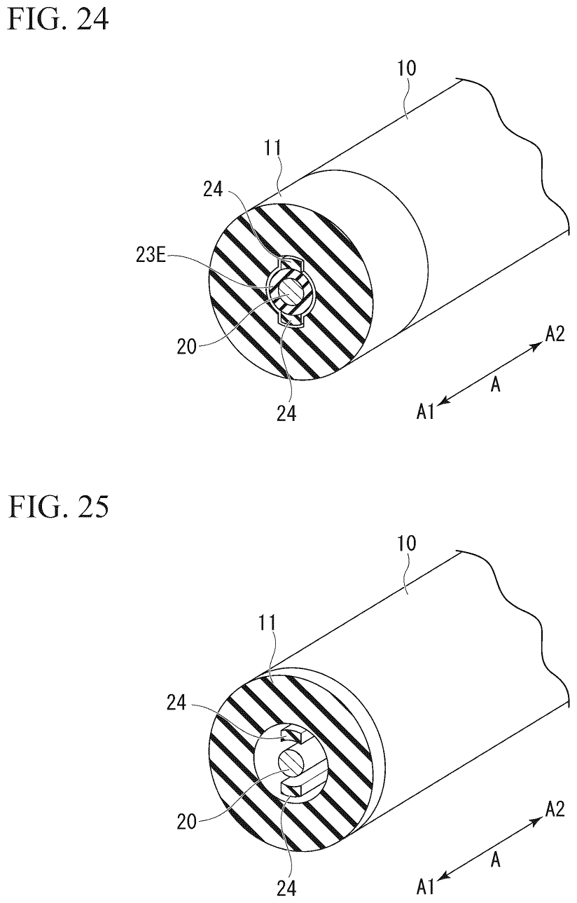

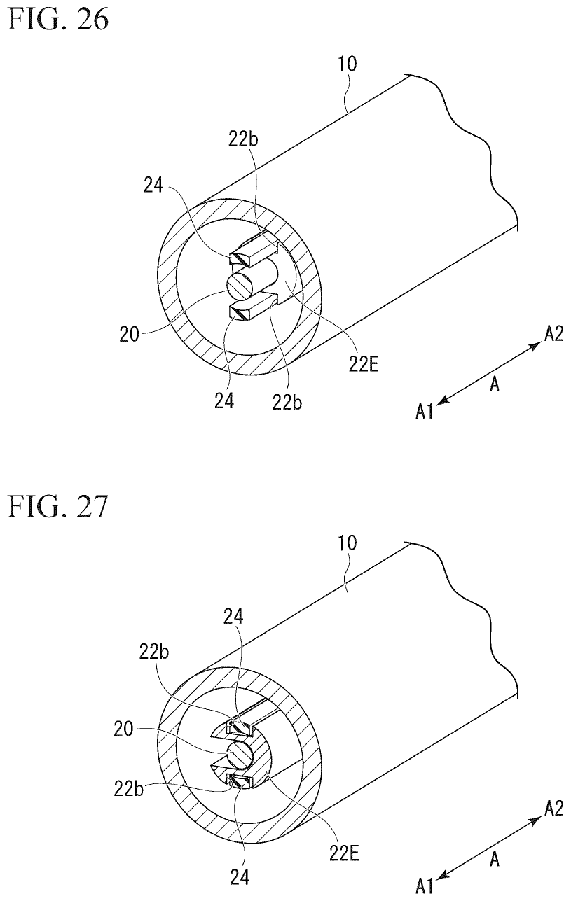

FIGS. 21 to 23 are example cross-sectional views of the distal end of the treatment tool 100E. FIG. 24 is an example of a cross-sectional view taken along line Y1-Y1 in FIG. 21. FIG. 25 is an example of a cross-sectional view taken along line Y2-Y2 in FIG. 21. FIG. 26 is an example of a cross-sectional view taken along line Y3-Y3 in FIG. 21. FIG. 27 is an example of a cross-sectional view taken along line Y4-Y4 in FIG. 21. The knife (electrode) 2E can be a metal member. The knife 2E can be formed of a material such as stainless steel. The knife 2E can be conductive, and high-frequency current can be passed through it. The knife 2E can include a rod 20, an electrode 21E, a connector 22E, an insulating member 23E, and a power transmission member 24.

The electrode 21E can be a disc-shaped conductive member provided at the distal end of the rod 20. In a front view seen from the direction along the longitudinal axis A, the outer periphery of the electrode 21E can be located inside the outer surface 31 of the insulating chip 3A in the radial direction R over the entire circumference.

The connector 22E can be a substantially cylindrical member made of metal. The connector 22E connects the rod 20 and the operating wire 4. The connector 22E and the rod 20 can advance and retract as the operating wire 4 advances and retracts. As shown in FIG. 26, the connector 22E can include an insertion passage 22b with a concave cross section provided on both sides of the rod 20 in the radial direction R.

The insulating member (insulating tube) 23E can be an insulating tube that covers the outer periphery of the rod 20. The insulating member 23C can be attached to the rod 20 so as to be freely movable in the longitudinal axis direction A along the outer periphery of the rod 20.

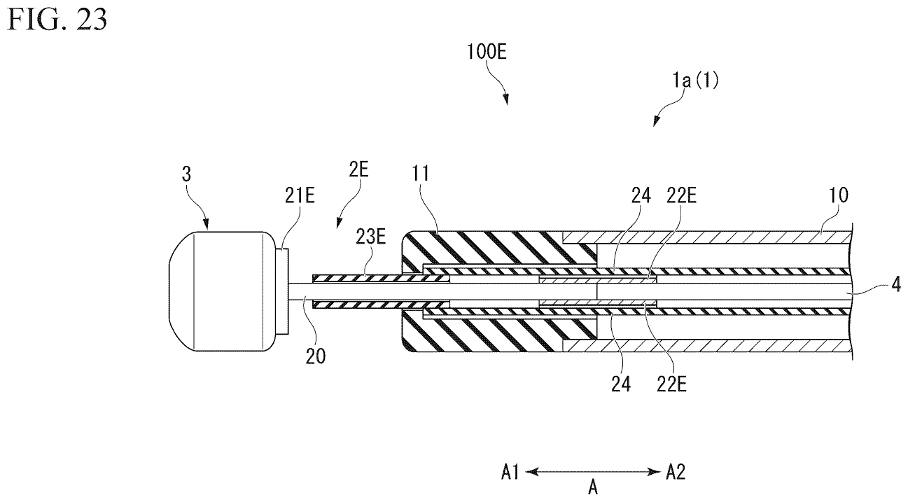

The power transmission member 24 can be or include a member that passes through the insertion passage 22b and can advance and retract in the longitudinal axis direction A. The distal end of the power transmission member 24 can be attached to the insulating member 23E. The proximal end of the power transmission member 24 can be connected to the lever 56 of the operating portion 5E. The power transmission member 24 can advance and retract relative to the operation wire 4, thereby causing the insulating member 23E to advance and retract relative to the rod 20. As shown in FIG. 21, the power transmission member 24 can advance relative to the operation wire 4 and the sheath 1 to a position where the distal end of the power transmission member 24 abuts against the electrode 21E. Also, as shown in FIG. 23, the power transmission member 24 can be configured to advance until it abuts against the distal end member 11 when the knife 2E is fully advanced. In this case, when the knife 2E is advanced to its maximum extent and the distal end of the power transmission member 24 can be advanced until it abuts against the distal end member 11, and a gap can be created between the proximal end surface of the electrode 21E and the distal end of the insulating tube 23E.

The operating portion 5E can include an operating portion main body 51, a slider 52, a power supply connector 53, and a lever 56.

The lever (slide lever) 56 can be attached to the slider 52 so that it can advance and retract. The proximal end of the power transmission member 24 can be attached to the lever 56. The power transmission member 24 can advance and retract by advancing and retracting the lever 56.

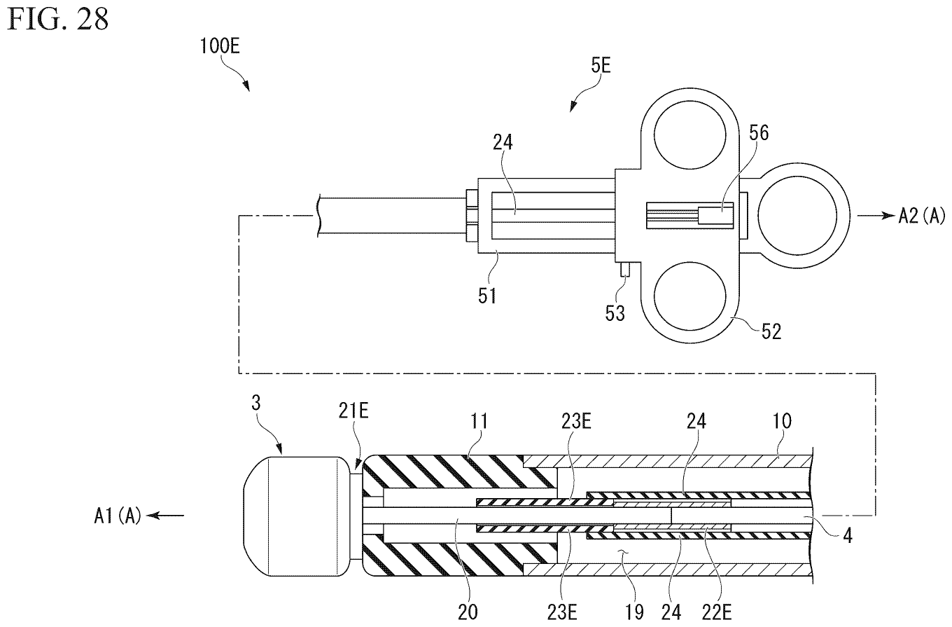

FIGS. 28 to 31 are example diagrams showing the movement of the knife 2E by operating the operating portion 5E.

As shown in FIG. 28, when the slider 52 and lever 56 are retracted, the distal end of the insulating member 23E can be positioned in the internal space 19 of the sheath 1. By retracting the lever 56 to its full extent, the insulating member 23E can be retracted until the distal end of the insulating member 23E is in a position inside the sheath 1.

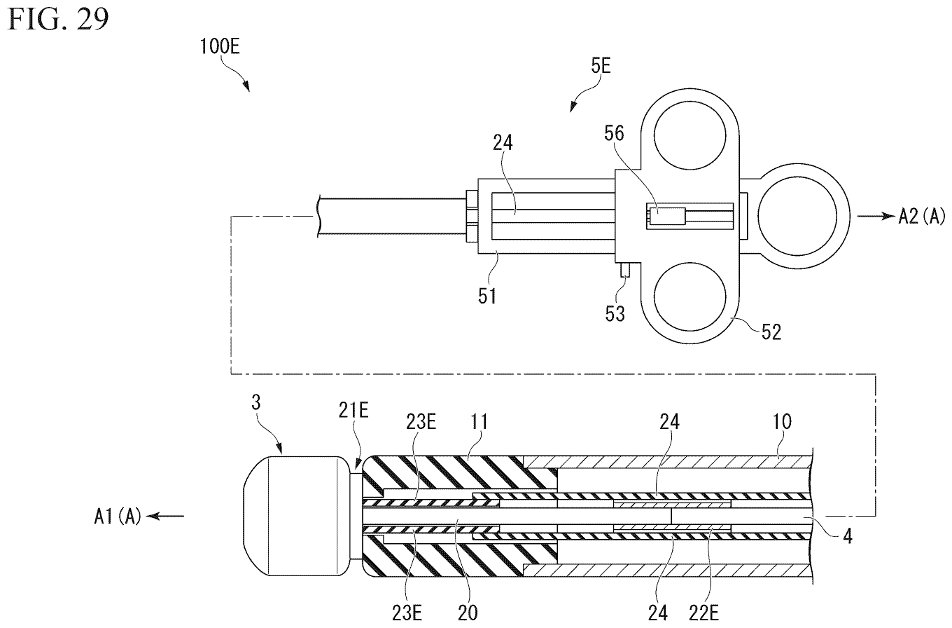

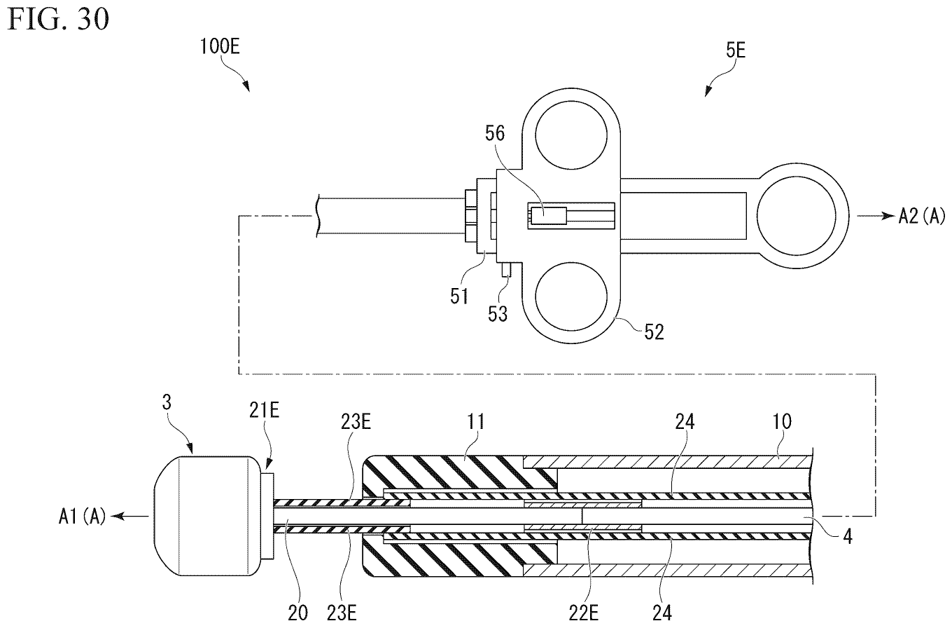

As shown in FIGS. 29 and 30, the surgeon can advance the rod 20 and the insulating member 23E together by advancing the slider 52 relative to the operating portion main body 51, thereby advancing the slider 52 and the lever 56 together.

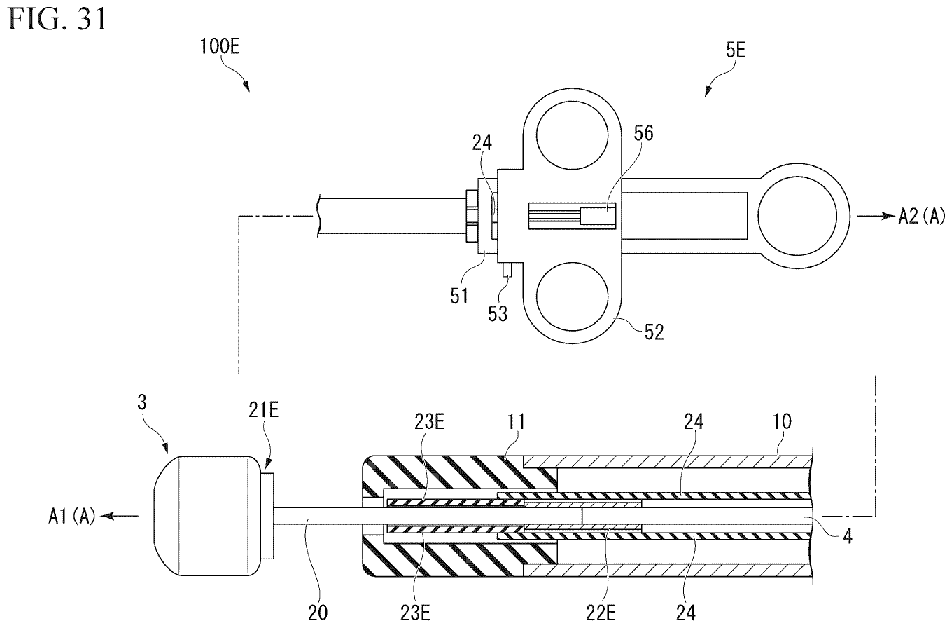

As shown in FIGS. 28 to 31, the surgeon can advance and retract the insulating member 23E relative to the rod 20 by advancing and retracting the lever 56 relative to the slider 52. As shown in FIG. 30, when the rod 20 is in the state of being fully advanced, by advancing the lever 56 to the maximum extent, the insulating member 23E can advance relative to the rod 20 to a position where the distal end of the insulating member 23E abuts against the electrode 21E. As shown in FIG. 31, when the rod 20 is in the state of being fully advanced, by retracting the lever 56 to the maximum extent, the insulating member 23E can retract relative to the rod 20 until the distal end of the insulating member 23E is located inside the sheath 1. In other words, the insulating member 23E can freely advance and retract between the position where the distal end of the insulating member 23E abuts against the electrode 21E and a position inside the sheath 1. The insulating member 23E can be movable between a position where the distal end of the insulating member 23E abuts against the electrode 21E and a position between the distal end of the electrode 21E and the distal end of the sheath 1 when the rod 20 is advanced to the maximum. The insulating member 23E can be movable between a position where the distal end of the insulating member 23E is disposed in the sheath 1 and a position where the distal end of the insulating member 23E is disposed between the electrode 21E and the distal end of the sheath 1 when the rod 20 is advanced to the maximum.

According to the treatment tool 100E according to this embodiment, the degree of exposure of the rod 20 can be adjusted by adjusting the position of the insulating member 23E according to the type of treatment and the condition of the affected area.

The third embodiment of the present disclosure has been described above in detail with reference to the drawings, but the specific configuration is not limited to this embodiment, and design changes within the scope of the present disclosure are also included. In addition, the components shown in the above-mentioned embodiment and modifications can be appropriately combined to configure the treatment tool.

The insulating chip 3 of the treatment tool 100E can have a tapered surface 33t, like the insulating chip 3A shown in FIG. 16. A front space (a gap) SF can be formed between the tapered surface 33t and the electrode 21E.

The present disclosure can be applied to endoscope treatment tools.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

What is claimed is:

1. An endoscope treatment tool, comprising:
a sheath;
a rod protruding from a distal end of the sheath, the rod being conductive;
an electrode connected to a distal end of the rod;
an insulator located distally relative to the electrode; and
an insulating member covering at least part of an outer circumferential surface of the rod.

2. The endoscope treatment tool according to claim 1, wherein the insulating member includes an insulating coating.

3. The endoscope treatment tool according to claim 2, wherein the insulating coating covers an entire surface of the outer circumferential surface of the rod.

4. The endoscope treatment tool according to claim 2, wherein the insulating coating covers a circumferential portion of the outer circumferential surface of the rod.

5. The endoscope treatment tool according to claim 1, wherein the insulating member includes an insulating tube.

6. The endoscope treatment tool according to claim 5, wherein the insulating tube is fixed to the rod.

7. The endoscope treatment tool according to claim 5, wherein the insulating tube is configured to advance and retract relative to the rod.

8. The endoscope treatment tool according to claim 7, wherein the insulating tube is configured to advance and retract between a position where a distal end of the insulating tube abuts against the electrode and a position between the electrode and the distal end of the sheath.

9. The endoscope treatment tool according to claim 7, wherein the insulating tube is configured to move between a position where a distal end of the insulating tube abuts against the electrode and a position within the sheath.

10. The endoscope treatment tool according to claim 1, wherein a distal end of the insulating member coincides with a proximal end of the electrode, and wherein the proximal end of the insulating member is located within the sheath or at the distal end of the sheath.

11. The endoscope treatment tool according to claim 1, wherein a distal end of the insulating member is in contact with a proximal end of the electrode, and wherein the proximal end of the insulating member is located within the sheath or at the distal end of the sheath.

12. The endoscope treatment tool according to claim 1, wherein at least part of the insulating member extends from the electrode to the distal end of the sheath.

13. The endoscope treatment tool according to claim 1, wherein the insulating member covers at least a portion of a proximal end side of the rod protruding from the sheath.

14. The endoscope treatment tool according to claim 1, wherein a proximal end of the insulator has a tapered surface narrowing toward the electrode, and wherein the tapered surface is spaced apart from the electrode in a longitudinal direction of the rod.

15. An endoscope treatment tool, comprising:
a sheath; and
a treatment portion protruding from a distal end of the sheath, wherein the treatment portion includes:
a first insulating member;
a second insulating member arranged on a proximal end side of the first insulating member, the second insulating member having an outer diameter smaller than an outer diameter of at least part of the first insulating member; and
an electrode located between the first insulating member and the second insulating member in a longitudinal direction of the treatment portion.

16. The endoscope treatment tool according to claim 15, wherein the first insulating member includes an insulating coating.

17. The endoscope treatment tool according to claim 15, wherein the first insulating member includes an insulating tube.

18. The endoscope treatment tool according to claim 15, wherein the first insulating member is configured to move between a position where a distal end of the first insulating member abuts against the electrode and a position between the electrode and a distal end of the sheath.

19. The endoscope treatment tool according to claim 15, wherein the first insulating member is configured to move between a position where a distal end of the first insulating member abuts against the electrode and a position within the sheath.

20. An endoscope treatment tool, comprising:
a sheath;
a rod protruding from a distal end of the sheath;
an electrode connected to a distal end of the rod; and
an insulator located distally relative to the electrode, wherein the endoscope treatment tool is configured so that electricity is not passed through tissue from at least part of an outer circumferential surface of the rod.
